(19) United States
(12) Patent Application Publication
(10) Pub. No.: US 2006/0264966 A1
(43) Pub. Date: Nov. 23, 2006

(54) Vertebroplasty leak prevention sleeve and method

(75) Inventor: William D. Armstrong, Memphis, TN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: MED Institute, Inc., West Lafayette, IN (US)

(21) Appl. No.: 11/430,100

(22) Filed: May 8, 2006

Related U.S. Application Data
(60) Provisional application No. 60/680,172, filed on May 12, 2005.

(51) Int. Cl. A61F 2/00

(52) U.S. Cl. 606/92

(57) ABSTRACT

A sleeve for use in inhibiting leakage of bone cement during a vertebroplasty procedure. The sleeve includes a hollow main body portion and a plunger-like shield disposed at the distal end of the main body portion. The main body portion is sized to be fitted over the cannula of a conventional vertebroplasty assembly through which the cement is injected, and the shield is sized to cover the injection hole in the vertebra, thereby inhibiting leakage of injected cement back through the injection hole.

### RELATED APPLICATION

The present patent document claims the benefit of the filing date under 35 U.S.C. §119(e) of Provisional U.S. Patent Application Ser. No. 60/680,172, filed May 12, 2005, which is hereby incorporated by reference.

### BACKGROUND

1. Technical Field. This invention relates generally to the field of vertebroplasty, and more particularly, to a sleeve for preventing leakage of cement from a vertebral body during a vertebroplasty procedure.

2. Background Information

Vertebroplasty is a minimally invasive nonsurgical procedure for treating compression fractures of the spinal vertebrae. Compression fractures of the spinal vertebrae normally result from conditions such as the collapse of a spinal vertebra. Although many conditions are known to cause vertebral collapse, the leading conditions are benign osteoporotic fractures, malignant metastatic disease, and benign tumors of the bone. Vertebral compression fractures often result in extreme pain for the patient, which may be accompanied with significant limitations on the patient's mobility.

Traditional treatments for compression fractures of this type have included bed rest, the administration of analgesics and/or muscle relaxants, and affixation of a brace to the affected area. In many cases, patients respond favorably to these traditional treatments. However, other patients are not so fortunate, and continue to be plagued with a high level of pain and/or a decrease of mobility. Thus, for these patients, the vertebroplasty procedure is performed in an attempt to restore the vertebral body as close as possible to its former size and shape, within the limitations of the procedure. In a successful outcome, the pain associated with the affliction is significantly reduced or eliminated, and the patient's mobility is at least substantially restored.

During a vertebroplasty procedure, a bone cement, such as polymethylmethacrylate (PMMA), is injected into the affected vertebra under X-ray guidance to fill empty spaces at the fracture site, generally along a transpedicular or posterolateral approach. The cement restores strength to the bone, and reduces the likelihood that it will fracture again. Generally, patients suffering from compression fracture regain mobility and achieve pain relief within hours of the conclusion of the procedure. Vertebroplasty procedures are now in widespread use, and details of the technique are well known to those skilled in the art. Further information concerning the vertebroplasty procedure and its benefits is provided in, e.g., Cotton, A., et al., “Percutaneous Vertebroplasty: State of the Art”, Radiographics, 1998 March-April: 18(2):311-323, and Predey T A, Sewall, L E, Smith S J. “Percutaneous Vertebroplasty: New Treatment For Vertebral Compression Fractures”, American Family Physician 2002; 66:611-615. These articles are incorporated by reference herein.

Although vertebroplasty has become a widely accepted technique for treating compression fractures, this procedure is not without potential complications. One particularly troublesome complication is the possibility of leakage of a portion of the injected bone cement from the injection site. Generally, such leakage occurs through the hole in the vertebral body into which the needle had been inserted, although some leakage may also occur through other holes or voids in the weakened bone structure. In most cases, leakage of a small amount of cement does not cause complications. However, in some cases, the cement can leak into a potentially dangerous area, such as the spinal canal. In other cases, it can migrate into the blood stream or the lungs. The leakage or migration of even a small amount of cement into such areas can have significantly adverse consequences to a patient. In still other cases, leaked bone cement can harden at inopportune locations, from which it can form a hardened body that may press on and disrupt nerve roots or the spinal cord.

Thus, even though vertebroplasty procedures generally are carried out without the leakage of bone cement, or with the leakage of insubstantial amounts of cement, there are instances where such leakage can be problematic for the patient. It would be advantageous if the vertebroplasty technique could be carried out in a manner that reduces the risk of leakage of bone cement, along with the adverse consequences that may accompany such leakage.

### BRIEF SUMMARY

The problems of the prior art are addressed by the apparatus and method of the present invention. In one form thereof, the present invention is directed to an improvement to a vertebroplasty assembly of the type including a cannula, an insert sized to be received in a lumen of the cannula, the insert having a needle tip for penetrating a vertebral body and forming a injection hole therein, and an injection device for injecting cement through the injection hole into the vertebral body via the cannula lumen. The improvement comprises a sleeve having a main body portion and a shield portion disposed at a distal end of the main body portion. The main body portion is sized to be fitted over at least a portion of the cannula. The shield portion is penetrable by a distal end of the cannula, and is sized to substantially cover the injection hole and prevent leakage therethrough when cement is injected into the vertebral body.

In another form thereof, the present invention is directed to an improvement to a method for performing vertebroplasty of the type wherein
cement is injected via a cannula into a vertebral body through an injection hole formed in the vertebral body. The improvement comprises fitting a sleeve over at least a distal portion of the cannula, wherein the sleeve has a main body portion and a shield disposed at a distal end of the main body portion, and wherein the shield is penetrable by the distal end of the cannula and is sized to substantially cover the injection hole. The sleeve is positioned such that the shield abuts the injection hole, thereby preventing leakage of cement from the vertebral body through the hole.

In still another form thereof, the invention is directed to a sleeve for use with a vertebroplasty assembly for preventing cement leakage during a vertebroplasty procedure, wherein the vertebroplasty assembly is of the type having a cannula and an injection device for injecting cement through the cannula into a vertebral body. The sleeve comprises a hollow main body portion, and a plunger disposed at a distal end of the main body portion. The main body portion is sized to be fitted over at least a portion of the cannula. The plunger is penetrable by a distal end of the cannula, and is sized to substantially cover an injection hole when cement is injected into the vertebral body.

### BRIEF DESCRIPTION OF THE DRAWINGS

FIG. 1 is an isometric view of a prior art vertebroplasty needle assembly;

FIG. 2 is a side view of the cannula portion of the prior art assembly of FIG. 1;

FIG. 3 is a side view of a stylet for use with the prior art assembly of FIG. 1;

FIG. 4 is side view of a vertebroplasty sleeve according to an embodiment of the present invention;

FIG. 5 is an end view of the vertebroplasty sleeve of FIG. 4, as viewed from the distal end of the sleeve;

FIG. 6 is a perspective view of the main body of the vertebroplasty sleeve of FIG. 4;

FIG. 7 is a perspective view of the plunger of the vertebroplasty sleeve of FIG. 4, having a portion removed to better illustrate its configuration; and

FIG. 8 is a side view of a conventional vertebroplasty needle assembly in combination with a vertebroplasty sleeve according to the present invention.

### DETAILED DESCRIPTION OF THE DRAWINGS AND THE PRESENTLY PREFERRED EMBODIMENTS

For purposes of promoting an understanding of the present invention, reference will now be made to the embodiments illustrated in the drawings, and specific language will be used to describe the same. It is nevertheless to be understood that no limitation of the scope of the invention is thereby intended, the proper scope of the invention being indicated by the claims appended below and the equivalents thereof. The figures are not all drawn to the same scale to avoid obscuring the details of the finer structures. The following detailed description of the preferred embodiments will make clear the preferred arrangement, size relationships and manner of using the components shown herein.

In the following discussion, the terms “proximal” and “distal” will be used to describe the opposing axial ends of the inventive apparatus, as well as the axial ends of various component features of the apparatus. The term “proximal” is used in its conventional sense to refer to the end of the apparatus (or component) that is handled by, or is closest to the operator during use of the vertebroplasty assembly. The term “distal” is used in its conventional sense to refer to the end of the apparatus (or component) that is initially inserted into the patient, or that is closest to the patient.

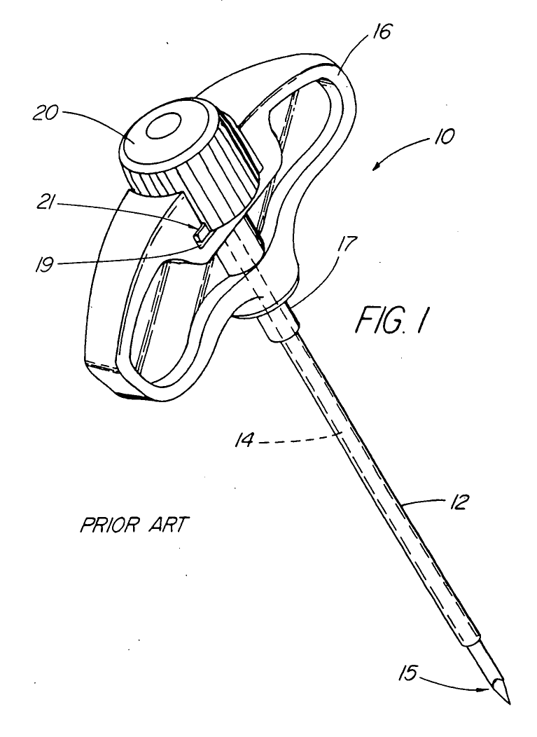

FIGS. 1-3 illustrate features of a conventional vertebroplasty needle assembly 10 for injecting bone cement or other suitable biomaterial into a vertebral body. Vertebroplasty needle assembly 10 generally consists of a cannula 12 having a lumen extending therethrough, and a stylet 14 removably received in the cannula lumen. The main body portion of the stylet 14 is shown in phantom in FIG. 1, and the exposed distal tip 15 is not in phantom. Typically, cannula 12 and stylet 14 are formed of surgical stainless steel, although those of skill in the art will recognize that the cannula and insert may be constructed of other suitable materials.

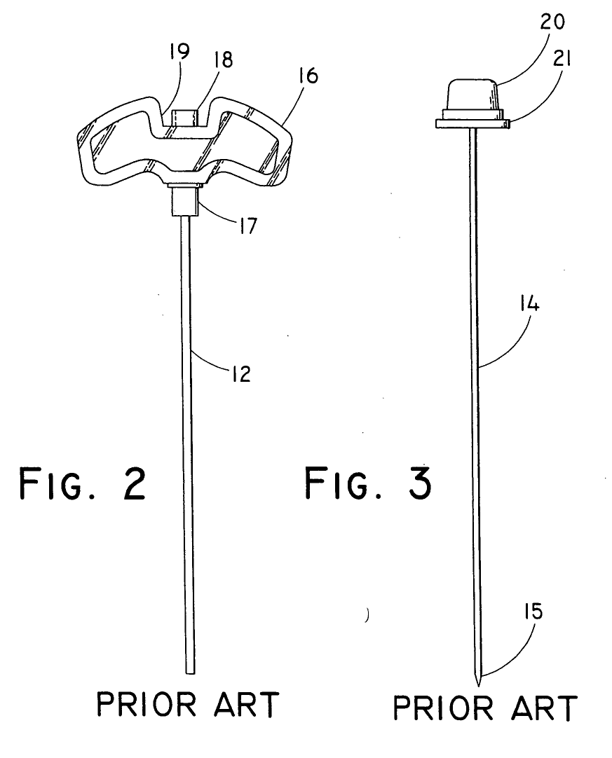

FIG. 2 illustrates cannula 12 removed from the remainder of assembly 10. As illustrated, a handle 16 is affixed to the proximal end of cannula 12. Handle 16 includes a passageway (not shown) extending longitudinally therethrough, and a hub 17 or similar receptacle for receiving the proximal end of the cannula. Preferably, an engagement member, such as threaded member 18, extends from a proximal end of handle 16 for engagement with the stylet, or with a syringe or other fluid conduit.

FIG. 3 illustrates a stylet 14 of a type that is used with prior art assembly 10. Stylet 14 includes a sharpened tip 15 at its distal end. Distal tip 15 may comprise a conventional trocar-shaped tip. Those skilled in the art are well aware that numerous different tip configurations are conventionally used in such needle assemblies, any of which may be substituted for the tip shown in a particular application. Typically, the needle configuration is matched with the distal end of the cannula such that a generally smooth, or continuous, transition is formed thereby. A connector, such as cap 20, is affixed to the proximal end of stylet 14. Cap 20 typically includes internal threads (not shown) for engagement with handle threaded member 18, and may include a tab 21 or similar structure for releasable engageable with handle 16 when stylet 16 is received within the lumen of cannula 14. In the embodiment shown, tab 21 is releasably receivable in a corresponding slot 19 formed in handle 16. When cap 20 and handle 16 are releasably engaged as shown in FIG. 1, the stylet 14 is received within the lumen of cannula 12, and the sharpened distal tip 15 of the stylet projects distally from needle assembly 10, as shown in FIG. 1.

During use of the conventional needle apparatus in a typical vertebroplasty procedure, the patient is initially placed in a prone position so that the affected vertebral body is within the field of an imaging device, such as an X-ray projection fluoroscopy imaging device. The skin overlying the vertebral body is prepped and draped in the usual manner utilizing acceptable sterile technique. A suitable anesthetic is injected into the periosteum of the pedicle to be entered, as well as into the surrounding area. Using a scalpel, a skin incision of about five millimeters is made, and the selected pedicle is initially entered. The leading (i.e., distal) end of the needle assembly is inserted into the incision, and passed down the selected pedicle until it enters the vertebral body.

The cement composition is mixed to the desired consistency, and loaded into a conventional cement injector. At this time, the engagement between stylet cap 20 and handle 16 is released, and stylet 14 is removed from the assembly. Removal of stylet 14 allows a cement injector, such as a syringe, to be attached to the assembly. FIG. 8 illustrates the attachment of a conventional syringe 26 to a vertebroplasty apparatus. The syringe is releasably attached to handle 16 by well-known means, such as by threadably attaching the distal end of the syringe to handle threaded member 18. The cement is thereafter injected into the vertebral body through the cannula in well-known manner. Preferably, an opacifier is added to the cement, so that the cement can be detected by the imaging device. If it is determined through X-ray visualization that a sufficient amount of cement has been injected to provide the desired strength to the vertebral body, then the treatment method is complete. If, however, it is determined that another injection of additional cement is required, the mixing and injection processes may be repeated, generally by injecting cement along the opposite pedicle from that used for the initial injection.

The vertebroplasty technique described above has now become fairly routine. However, the technique remains problematic, due in part to the risk of bone cement leakage out of the pedicle(s). When bone cement leakage occurs, most such leakage occurs through the hole in the vertebral body through which the injection had been made. In order to address this cement leakage problem, the present invention comprises an apparatus for use in connection with a conventional vertebroplasty needle assembly. Use of the inventive apparatus prevents the leakage of most, if not all, of the bone cement that may otherwise leak through the affected vertebral body.

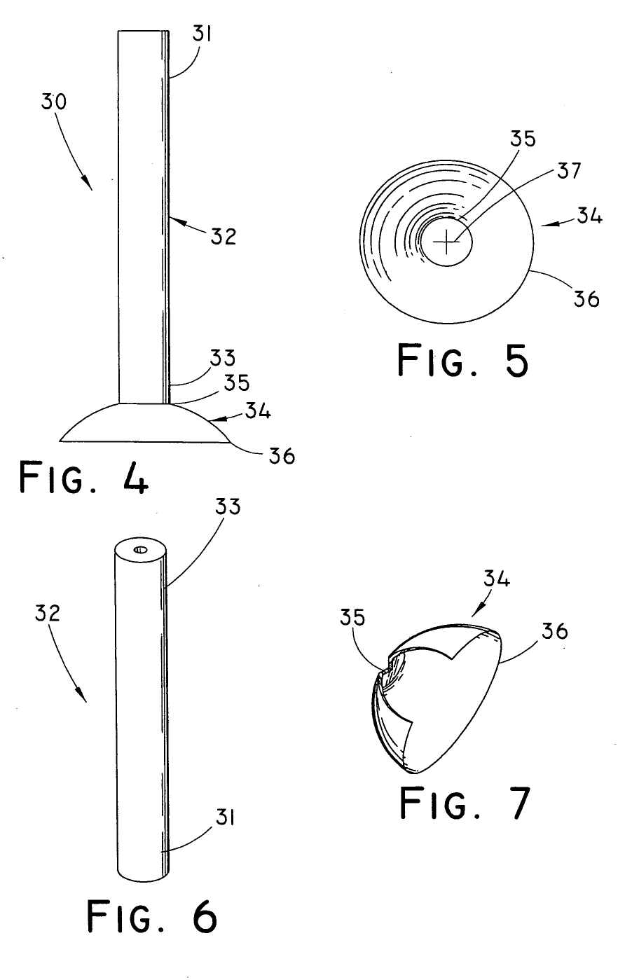

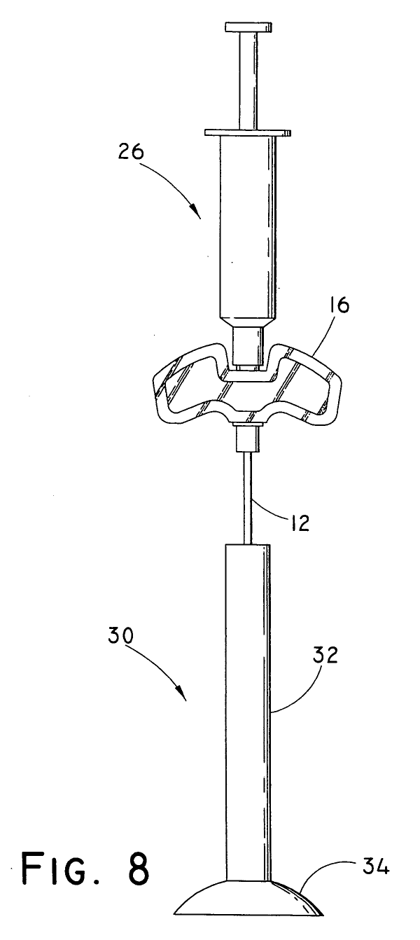

In a preferred embodiment, the apparatus comprises a sleeve 30 that is sized to fit over the distal end of a conventional needle assembly during the injection of cement into the vertebral body. One embodiment of sleeve 30 is illustrated in FIGS. 4 and 5. As illustrated, sleeve 30 includes a hollow generally cylindrical main body portion 32, and a larger diameter shield member 34 positioned at the distal end of main body portion 32. Preferably, the shield member is configured in the nature of a plunger. Main body portion 32 has a proximal portion 31 and a distal portion 33. Plunger 34 comprises a smaller diameter proximal portion 35 and a larger diameter distal portion 36. Main body portion 32 is further illustrated in FIG. 6, and plunger 34 is further illustrated in FIG. 7. A portion of plunger 34 has been removed from FIG. 7 to better illustrate its configuration. Plunger 34 can be affixed to distal portion body 33 by any technique known in the art, such as by adhesion, a snap fit, a threaded connection, etc.

Main body portion 32 may be formed of conventional materials used in the medical arts. Although main body portion 32 need not be rigid, it is preferred that the body have sufficient rigidity such that is does not yield, or yields only in minor amount, when utilized in a vertebroplasty procedure. Non-limiting examples of suitable materials for forming main body 32 include conventional metals and metal alloys, such as stainless steel, and rigid or semi-rigid biocompatible polymeric materials, such as PTFE, polycarbonates, nylon, polypropylene, PEEK, and acetal. Preferably, plunger 34 comprises an elastic material that is configured to be affixed at the terminal end of distal portion 33 of the main body portion of the sleeve. Non-limiting examples of materials that may be utilized to form plunger 34 include various elastomeric rubbers and polymers known in the art to have such capability, such as silicone elastomers, polyisoprene, styrene, polyurethanes, and various thermoplastic elastomers, such as KRATON®.

Plunger small diameter portion 35 is close-ended, that is, it spans the distal end of hollow main body portion 32 when sleeve 30 is assembled as shown in FIG. 4. As a result, when plunger 34 is affixed to the distal end of the main body portion, it forms a seal at distal end 33. Plunger large diameter portion 36 is open-ended. Small diameter portion 35 may be provided with pre-scored portion 37 as shown in FIG. 5, for facilitating the passage of needle tip 15 and the distal end of cannula 12 through the seal defined by small diameter portion 35. Thus, when a needle tip and/or a cannula end are passed through sleeve 30 during a vertebroplasty procedure, small diameter portion 35 yields to the entry of the needle tip. Following removal of the needle, portion 35 re-conforms, or substantially re-conforms, to the sealed condition.

Although it is preferred to form plunger 34 of an elastomeric composition, this is not required, and the plunger can alternatively comprise other compositions, such as the more rigid compositions used to form main body portion 32. Furthermore, main body portion 32 and plunger 34 need not be initially formed from separate components and affixed as described. Rather, sleeve 30 can be formed as an integral composition that includes the main body portion and the plunger. As a still further alternative, plunger 34 can be formed from a rigid frame member, with an elastomeric composition forming the close-ended side. In any of these arrangements, however, the plunger must be structured in a manner such that the needle can penetrate the close-ended portion, and that this portion is capable of at least substantially re-sealing following withdrawal of the needle. Those skilled in the art can readily select appropriate molding or other fabricating techniques to form the separate components or the integral structure described herein.

Use of the apparatus of the present invention in a vertebroplasty procedure thus requires only relatively small modifications to the conventional procedure previously described. For example, prior to insertion of the needle into the affected area of the patient, the sleeve 30 is inserted over the distal end of the needle assembly, as shown in FIG. 8. As the needle tip passes through the small diameter portion 35 and penetrates the affected vertebral body, the sleeve plunger 34 is in substantial abutment with the outer portion of the vertebral body. Following attachment of syringe 26 to the assembly and the injection of the cement through the cannula into the vertebral space in the conventional manner, the plunger 34 prevents cement from leaking back through the injection hole.

Once all of the cement has been injected, sleeve 30 is left in place until the cement hardens sufficiently such that further migration back through the injection hole is unlikely. Typically, this time period does not exceed about twenty minutes, and in many cases will be about 8-10 minutes, and in some cases as short as 4-5 minutes. Those skilled in the art will appreciate that the rate of hardening of bone cement is dependent on factors such as the type and consistency of the cement, and the amount of cement injected into the vertebral body. Thus, the period of time in which the plunger remains abutting the vertebral body may be increased, or decreased, according to the particular parameters used in an individual case.

It is known that most leakage of cement in a vertebroplasty procedure occurs through the original needle hole. By maintaining plunger 34 in position against the vertebral body, the plunger acts as a shield, so that the bone cement cannot leak back through the hole. As a result, the cement remains in its intended position in the affected vertebral body until it hardens sufficiently such that the sleeve can thereafter be removed. If cement is injected through both the left and right pedicles, then a separate sleeve 30 may be used with each of the separate injection apparatuses if desired.

The size of main body portion 32 and plunger 34 of sleeve 30 can vary depending on the size of the components of the needle apparatus. The diameter and length of the sleeve is preferably sized such that it is compatible with the gauge and the length of the needle and cannula used for the vertebroplasty procedure. One suitable biopsy needle for this indication is marketed by William Cook Europe, and is a 13 gauge needle with a length of 10 cm. Other suitable components of a vertebroplasty assembly are commercially available as OSTEO-SITE® bone access products, marketed by Cook Incorporated, of Bloomington, Ind. One example of a suitable sleeve that may be used with conventional vertebroplasty assemblies includes a plunger having a major diameter in the range of about 5-15 mm, and a height/depth of about 3-5 mm. Those skilled in the art will appreciate that needles and sleeves of varying dimensions may be used in an appropriate case, depending primarily on the size of the patient and the nature of the vertebroplasty procedure that is to be performed.

It is therefore intended that the foregoing detailed description be regarded as illustrative rather than limiting, and that it be understood that it is the following claims, including all equivalents, that are intended to define the spirit and scope of this invention.

1. In a vertebroplasty assembly of the type including a cannula, an insert sized to be received in a lumen of the cannula, the insert having a needle tip for penetrating a vertebral body and forming a injection hole therein, and an injection device for injecting cement through said injection hole into the vertebral body via said cannula lumen, the improvement comprising:
a sleeve having a main body portion and a shield portion disposed at a distal end of said main body portion, said main body portion sized to be fitted over at least a portion of the cannula, said shield portion being penetrable by a distal end of said cannula and being sized to substantially cover said injection hole and prevent leakage therethrough when cement is injected into the vertebral body.

2. The vertebroplasty assembly of claim 1, wherein said main body portion comprises a generally cylindrical body, and said shield portion comprises a plunger.

3. The vertebroplasty assembly of claim 2, wherein said plunger comprises a smaller diameter proximal portion and a larger diameter distal portion, said smaller diameter proximal portion configured for sealing a distal end of said generally cylindrical body.

4. The vertebroplasty assembly of claim 3, wherein said plunger comprises a generally elastomeric composition.

5. The vertebroplasty assembly of claim 4, wherein said plunger smaller diameter proximal portion includes a pre-scored portion.

6. The vertebroplasty assembly of claim 2, wherein said main body portion comprises a biocompatible metal or metal alloy.

7. The vertebroplasty assembly of claim 6, wherein said main body portion comprises stainless steel.

8. In a method for performing vertebroplasty of the type wherein cement is injected via a cannula into a vertebral body through an injection hole formed in said vertebral body, the improvement comprising:
fitting a sleeve over at least a distal portion of the cannula, the sleeve having a main body portion and a shield disposed at a distal end of the main body portion, the shield being penetrable by the distal end of the cannula and being sized to substantially cover the injection hole; and
positioning the sleeve such that the shield abuts the injection hole, thereby preventing leakage of cement from the vertebral body through the hole.

9. The method of claim 8, wherein the main body portion comprises a generally cylindrical body, and the shield comprises a plunger.

10. The method of claim 9, wherein the plunger comprises a smaller diameter proximal portion and a larger diameter distal portion, the smaller diameter proximal portion configured for sealing a distal end of the main body portion.

11. The method of claim 10, wherein the plunger comprises a generally elastomeric composition.

12. The method of claim 10, wherein the smaller diameter proximal portion includes a pre-scored portion for passage of the cannula therethrough.

13. The method of claim 10, wherein the main body portion comprises a member selected from the group consisting of metals, metal alloys, semi-rigid polymers and rigid polymers.

14. The method of claim 13, wherein the main body portion comprises stainless steel.

15. A sleeve for use with a vertebroplasty assembly for preventing cement leakage during a vertebroplasty procedure, the vertebroplasty assembly of the type including a cannula and an injection device for injecting cement through the cannula into a vertebral body, the sleeve comprising:
a hollow main body portion; and
a plunger disposed at a distal end of the main body portion, the main body portion sized to be fitted over at least a portion of the cannula, the plunger being penetrable by a distal end of the cannula and sized to substantially cover an injection hole in said vertebral body when cement is injected therein.

16. The sleeve of claim 15, wherein said plunger comprises a smaller diameter proximal portion and a larger diameter distal portion, said smaller diameter proximal portion configured for sealing a distal end of said main body portion.

17. The sleeve of claim 16, wherein said plunger comprises a generally elastomeric composition

18. The sleeve of claim 16, wherein said plunger smaller diameter proximal portion includes a pre-scored portion.

19. The sleeve of claim 15, wherein said main body portion comprises a biocompatible metal or metal alloy.

20. The sleeve of claim 19, wherein said main body portion comprises stainless steel.
